(19) United States
(12) Patent Application Publication
(10) Pub. No.: US 2020/0078035 A1
(43) Pub. Date: Mar. 12, 2020

(54) IMPLANT EXCISION AID

(71) Applicant: Merck Sharp & Dohme Corp., Rahway, NJ (US)

(72) Inventor: Brad Thomas Britland, Lake Hopatcong, NJ (US)

(73) Assignee: Merck Sharp & Dohme Corp., Rahway, NJ (US)

(21) Appl. No.: 16/533,989

(22) Filed: Aug. 7, 2019

Related U.S. Application Data
(63) Continuation of application No. 62/728,114, filed on Sep. 7, 2018.

(51) Int. Cl. A61B 17/30; B33Y 80/00

(52) U.S. Cl. CPC A61B 17/30; A61B 2017/00907; A61B 2017/305; B33Y 80/00

(57) ABSTRACT

An implant excision aid comprising a first plate comprising an external surface, an internal surface, a top portion, a bottom portion, a right side and a left side, wherein the bottom portion of the first plate is in communication with the patient's skin during use; a second plate comprising an external surface and an internal surface, a top portion, a bottom portion, a right side and a left side, wherein the bottom portion of the second plate is in communication with the patient's skin during use, wherein at least a portion of the bottom portion of the first or second plate or the internal surface of the first plate or second plate comprises a polymer or a metal, wherein the polymer or the metal generates enough friction so that the portion of the bottom of the first or second plate or the internal surface of the first plate or second plate is capable of moving the patient's skin during use; and a hinge wherein the hinge connects the top of the first plate to the top of the second plate.

### TECHNICAL FIELD

The present invention is directed to an implant excision aid for aiding in the removal of subdermal or subcutaneous, palpable implant. Specifically, the implant excision aid described herein is designed to make implant removal easier for the implant remover, usually a physician or medical caregiver, and to reduce technique variability from implant remover to implant remover.

### BACKGROUND

Subdermal or subcutaneous implants are routinely used for therapeutic value pertaining to adherence. Products such as IMPLANON®, NEXPLANON®, PROBUPHINE®, EVERSENSE®, VANTAS®, JADELLE®, SUPPRELIN® and NALTREXONE® are inserted subdermally or subcutaneously using application devices designed to insert them in the proper anatomical location and orientation and at the recommended depth, typically to achieve long-acting drug release.

At some point all non-biodegradable implants must be excised from the patient. Usually removal of the implant is at the conclusion of the useful life of the implant, but under certain circumstances, the implant might have to be removed prematurely. Excising of an implant requires first locating the implant by palpation, then manipulating the implant under the skin to cause “popping” of the implant up toward the surface of the skin which results in “tenting” of the skin above the implant. Once the implant is in this position, the implant remover makes an incision proximal to the location of the implant where the skin is tented, and then, preferably while continuing to ‘pop-up’ the implant, withdraws the implant with forceps.

The technique of manual popping of the implant toward the skin to achieve tenting requires a great deal of skill which is acquired through training and practice. Unfortunately, variation in remover to remover skill level contributes to inconsistencies in the process of positioning the implant for making the incision. Medical caregivers, such as physicians, who do not often perform this procedure often may not feel confident or comfortable performing this procedure. Moreover, some medical caregivers consider the current practice of popping as being awkward and in some instances painful to perform. The current practice of popping, to support/perform implant excision, can also be time-consuming and a resource burden, sometimes requiring a second person.

A means of standardizing the procedure of positioning the implant for removal can eliminate the need for training and reduce the level of skill and practice needed for successful removal of implants. To remedy this, described herein is an excision aid that potentially shortens the excision procedure duration, reduces the amount of training needed to acquire the skill for the procedure, making it easier for the remover to gain procedure proficiency, reduces the risk for contamination at or near the incision site and reduces technique variability from remover to remover.

### SUMMARY

Described herein are implant excision aids for removal of a subdermal or subcutaneous, palpable implant from a patient comprising a first plate comprising an external surface, an internal surface, a top portion, a bottom portion, a right side and a left side, wherein the bottom portion of the first plate is in communication with the patient's skin during use; a second plate comprising an external surface and an internal surface, a top portion, a bottom portion, a right side and a left side, wherein the bottom portion of the second plate is in communication with the patient's skin during use, wherein at least a portion of the bottom portion of the first or second plate or the internal surface of the first plate or second plate comprises a polymer or a metal, wherein the polymer or the metal generates enough friction so that the portion of the bottom of the first or second plate or the internal surface of the first plate or second plate is capable of moving the patient's skin during use; and a hinge wherein the hinge connects the top portion of the first plate to the top portion of the second plate.

In certain embodiments, the implant excision aids described herein have a first plate, wherein the first plate further comprises a window located at the bottom portion of the first plate. In certain embodiment, the window and the bottom portion of the first plate are adjacent and/or in contact with the patient's skin during use.

In certain embodiments, the implant excision aids described herein have a first plate and a second plate wherein, at least a portion of the internal surface of the first plate or second plate further comprises a textured surface, wherein, during use, the texture on the surface increases the friction between the bottom portion of the first or the bottom portion of the second plate or the internal surface of the first plate or the internal surface of the second plate, and the patient's skin. In certain embodiments described herein, the textured surface located on at least a portion of the bottom portion or the internal surface of the first plate or the bottom portion or the internal surface of second plate comprises a pattern of protrusions. In certain embodiments described herein, the textured surface located on at least a portion of the bottom portion or the internal surface of the first plate or the bottom portion or internal surface of the second plate comprises one or more ribs. In certain embodiments described herein, the ribs are in a pattern that is parallel to the hinge. In certain embodiments described herein, the ribs are in a pattern that is oblique to the hinge. In certain embodiments described herein, the ribs are in a pattern comprising a combination of oblique and parallel orientations relative to the hinge. In certain embodiments described herein, the ribs are curved.

In certain embodiments, the implant excision aids described herein have a first plate and a second plate wherein, the first and the second plates are generally planar. In other embodiments, the first and the second plate are generally curved. In yet other embodiments, the first and the second plates independently comprise a combination of planar and curved elements.

In certain embodiments, the hinge of the implant excision aids described herein comprises a living hinge. In certain embodiments, the hinge comprises an integral hinge pin and a receptacle. In certain embodiments, the hinge comprises an integrally molded flexible element. In certain embodiments, the hinge of the implant excision aids described herein comprises a hinge pin connecting the first and second plates.

In certain embodiments, the implant excision aids described herein, further comprise a ratcheting system, wherein the ratcheting system comprises at least one ratcheting arm located on the upper portion of the first or second plate, wherein each ratcheting arm comprises at least one tooth; and at least one opening located on the upper portion of the opposite plate, wherein the opening is capable of receiving the ratcheting arm as the ratcheting arm moves forward and wherein, the tooth on the ratcheting arm interacts with the edge of the opening to prevent the ratcheting arm from moving backwards.

In certain embodiments, the implant excision aids described herein, comprise a coil-type compression spring connecting the first and second plate. In other embodiments, the implant excision aids described herein, comprise one or more leaf springs connecting the first and second plate.

In certain embodiments, the implant excision aids described herein, have a first or second plate, wherein at least a portion of the bottom of the first or second plate or the internal surface of the first plate or second plate comprises polypropylene.

Also described herein are methods for aiding in the removal of a subdermal or subcutaneous implant from a patient using the implant excision aids described herein, comprising contacting a patient's skin with the bottom of the first and second plate, wherein the majority of the implant is located between the internal surfaces of the first and second plate of the excision aid; exerting a force on the excision aid wherein the excision aid locally displaces the proximal end of the implant toward the skin surface, causing tenting of the patient's skin. In certain embodiments, the method further includes, moving the skin in the distal direction to exaggerate the tenting of the skin, facilitate creating an incision in the skin and extracting the implant.

Also described herein, are processes of making or manufacturing the implant excision aids described herein. In certain embodiments, described herein is a process of making an implant excision aid, wherein the implant excision aid is injection molded from plastics. In certain embodiments, described herein is a process of making an implant excision aid, wherein the excision aid is machined from plastics. In certain embodiments, described herein is a process of making an implant excision aid, wherein the implant excision aid is machined from metal. In certain embodiments, described herein is a process of making an implant excision aid described herein, wherein the implant excision aid is printed using a 3D printer.

### FIGURES

FIG. 1 shows an embodiment of an implant excision aid described herein.

FIG. 2 shows an embodiment of an implant excision aid being used on the upper arm region of a patient.

FIG. 3 shows an embodiment of an implant excision aid being used on the skin of a patient.

FIG. 4 shows an embodiment of an implant excision aid being used on the skin of a patient.

FIG. 5 shows an embodiment of an implant excision aid being used on the skin of a patient.

FIG. 6 shows an embodiment of an implant excision aid being used on the skin of a patient.

FIG. 7 shows an embodiment of an implant excision aid described herein.

FIG. 8 shows an embodiment of an implant excision aid described herein.

FIG. 9 shows an embodiment of an implant excision aid described herein.

FIG. 10 shows an embodiment of an implant excision aid described herein.

FIG. 11 shows an embodiment of an implant excision aid described herein.

FIG. 12 shows an embodiment of an implant excision aid described herein.

FIG. 13 shows an embodiment of an implant excision aid described herein.

FIG. 14 shows an embodiment of an implant excision aid described herein.

FIG. 15 shows an embodiment of an implant excision aid described herein.

FIG. 16 shows an embodiment of an implant excision aid described herein.

FIG. 17 shows a magnified view of an embodiment of a protrusion that can be located on the internal surface of an embodiment of an implant excision aid described herein.

FIG. 18 shows a magnified view of an embodiment of a protrusion that can be located on the internal surface of an embodiment of an implant excision aid described herein.

FIG. 19 shows a magnified view of an embodiment of a protrusion that can be located on the internal surface of an embodiment of an implant excision aid described herein.

FIG. 20 shows an embodiment of an implant excision aid described herein.

FIG. 21 shows an embodiment of an implant excision aid described herein.

FIG. 22 shows a cross-section of a rib that can be located on the internal surface of an embodiment of an implant excision aid described herein.

FIG. 23 shows a cross-section of a rib that can be located on the internal surface of an embodiment of an implant excision aid described herein.

FIG. 24 shows a cross-section of a rib that can be located on the internal surface of an embodiment of an implant excision aid described herein.

FIG. 25 shows a cross-section of a rib that can be located on the internal surface of an embodiment of an implant excision aid described herein.

FIG. 26 shows a hinge that can be included in an embodiment of an implant excision aid described herein.

FIG. 27 shows a hinge that can be included in an embodiment of an implant excision aid described herein.

FIG. 28 shows a hinge that can be included in an embodiment of an implant excision aid described herein.

FIG. 29 shows a hinge that can be included in an embodiment of an implant excision aid described herein.

FIG. 30 shows a ratchet system that can be included in an embodiment of an implant excision aid described herein.

FIG. 31 shows a ratchet system that can be included in an embodiment of an implant excision aid described herein, wherein the ratcheting arms have not been threaded through their complementary openings.

FIG. 32 shows a ratchet system that can be included in an embodiment of an implant excision aid described herein, wherein the ratcheting arms have been threaded through their complementary openings.

FIG. 33 shows a magnified cross-section view of the ratchetting arm in its complementary opening of a ratchet system that can be included in an embodiment of an implant excision aid described herein.

FIG. 34 shows an embodiment of an implant excision aid described herein with a coil-type compression spring.

FIG. 35 shows an embodiment of an implant excision aid described herein with a coil-type compression spring.

FIG. 36 shows an embodiment of an implant excision aid described herein with a coil-type compression spring.

FIG. 37 shows an embodiment of an implant excision aid described herein with integrated springs.

FIG. 38 shows an embodiment of an implant excision aid described herein with a leaf-type spring.

FIG. 39 shows one possible way of positioning an excision aid described herein over an implant.

FIG. 40 shows one possible way of positioning an excision aid described herein over an implant.

### DETAILED DESCRIPTION

### Definitions

For the purposes of clarity and consistency, the term “proximal” refers to a direction away from the body of the patient and towards the implant remover. The term “distal” refers to a direction towards the body of the patient and away from the implant remover.

The term “implant”, refers herein to any subdermal or subcutaneous implant. In certain embodiments the implant contains etonogestrel. In certain embodiments the implant is NEXLANON® or IMPLANON NXT®.

The term “popping” of the implant, as used herein means, to locally displace the proximal end of the implant toward the skin's surface.

The term “tenting” of the skin, as used herein means, applying a force that causes a portion of the skin to take on a triangular or tent-like appearance.

Described herein is an implant excision aid for simplifying and standardizing the procedure for preparing the skin for excision of a subdermal or subcutaneous implant and for incising the skin to expose and remove the implant. The implant targeted for removal may have any shape. For example, most implants will be cylindrical in shape. However, the implant excision aids described herein can be used to aid in the removal of non-cylindrically shaped implants. Additionally, the targeted implant may have a wide range of width/length ratios. In certain embodiments described herein, the implant excision aids described herein are used to aid in the removal of a polymeric cylinder implant, measuring 2 mm in diameter and 40 mm long. In certain embodiments the implant is NEXLANON® or IMPLANON NXT®.

In certain embodiments the implant is non-biodegradable. In certain embodiments, the implant is palpable i.e. able to be felt.

Embodiments of the invention are discussed in detail below. In describing embodiments, specific terminology is employed for the sake of clarity. However, the invention is not intended to be limited to the specific terminology so selected. All references cited herein are incorporated by reference as if each had been individually incorporated.

A preferred embodiment of the invention is discussed in detail below. While specific exemplary embodiments are discussed, it should be understood that this is done for illustration purposes only. A person skilled in the relevant art will recognize that other components and configurations can be used without parting from the spirit and scope of the invention.

Described herein are implant excision aids for removal of a subdermal or subcutaneous implant from a patient comprising a first plate comprising an external surface, an internal surface, a top portion, a bottom portion, a right side and a left side, wherein the bottom portion of the first plate is in communication with the patient's skin during use; a second plate comprising an external surface and an internal surface, a top portion, a bottom portion, a right side and a left side, wherein the bottom portion of the second plate is in communication with the patient's skin during use, wherein at least a portion of the bottom portion of the first or second plate or the internal surface of the first plate or second plate comprises a polymer or a metal, wherein the polymer or the metal generates enough friction so that the portion of the bottom of the first or second plate or the internal surface of the first plate or second plate is capable of moving the patient's skin during use or alternatively, capable of applying a force to the implant to cause the proximal end of the implant to move toward the surface of the skin to cause tenting of the skin; and a hinge wherein the hinge connects the top portion of the first plate to the top portion of the second plate.

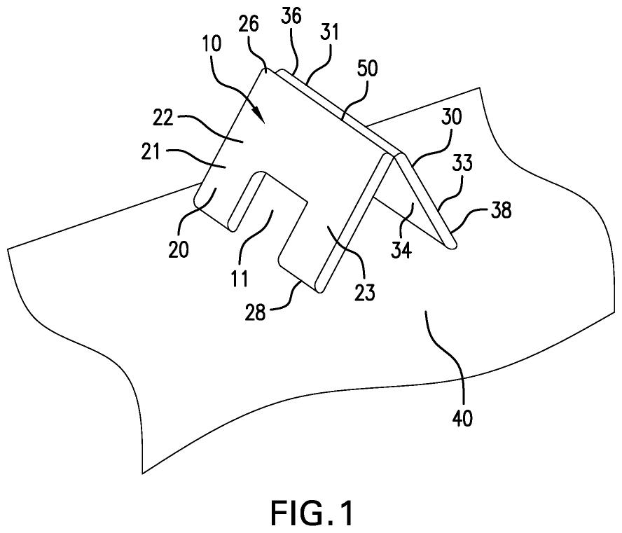

FIG. 1 shows an embodiment of the implant removal or excision aid described herein. The implant excision aid 10 is an implant removal or excision tool or aid comprising two plates, a first or proximal plate 20 and a second or distal plate 30. The first or proximal has an external surface 22, an internal surface (not shown), a top portion 26, a bottom portion 28, a right side 23 and a left side 21, wherein the bottom portion 28 of the first plate is in communication with the patient's skin 40 during use. The distal or second plate has an external surface (not shown) and an internal surface 34, a top portion 36, a bottom portion 38, a right side 33 and a left side 31, wherein the bottom portion 38 of the second plate is in communication with the patient's skin 40 during use.

The top portion of the first plate 26 and the top portion of the second plate 36 are joined together to form a hinge 50. As shown in the embodiment of FIG. 1, both the first 20 and second plates 30 are generally planar in shape.

In the embodiment shown in FIG. 1, the first plate 20 has a window 11 located on the bottom portion 28 of the first plate 20.

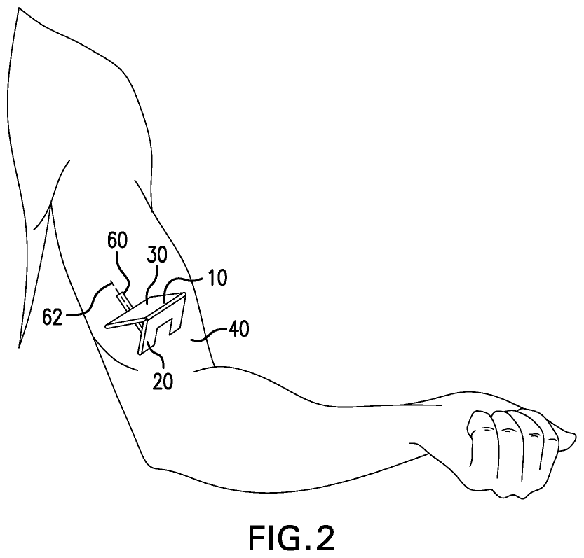

In the embodiment of the excision aid shown in FIG. 2, the implant excision aid 10 is positioned on a portion of a patient's skin 40 wherein the majority of the implant 60 is between the first plate 20 and second plate 30 of the excision aid 10 and is generally aligned with the axis 62 of implant 60. In FIG. 2, the implant excision aid 10 is shown being used to aid in the excision of an implant 60 inserted in the upper arm of the patient. However, one skilled in the art would know that a variety of such implants exist and may be inserted in other areas of the body. Moreover, the scaling of the size of the implant excision aid could be adjusted to the relative size of any implant or any implant features, to enable the implant excision aid to be used to excise implants from other areas of the body.

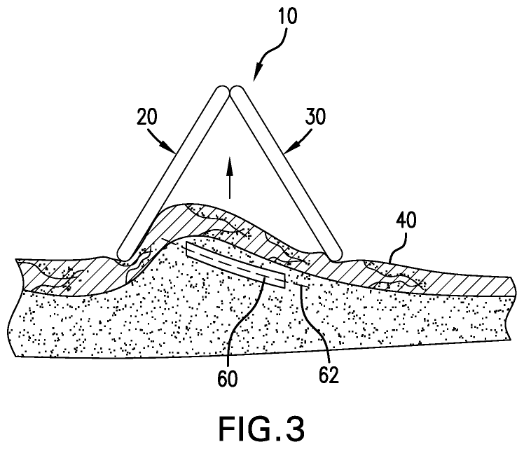

In the embodiment of the excision aid shown in FIG. 3, the implant excision aid 10 is configured to facilitate popping of the implant 60 to cause tenting of the patient's skin 40 in the region adjacent to the end of implant 60 and generally aligned with the axis 62 of implant 60, as shown in FIG. 3.

In the embodiment of the excision aid shown in FIG. 4, the implant excision aid 10 is placed on the patient's skin 40, wherein the bottom 28 of the first plate 20 and the bottom 38 of the second plate 30 are in contact with the patient's skin 40. In the embodiment shown in FIG. 4, the hinge 50 is generally aligned over and perpendicular to the end of implant 60, and a window 11, located on the bottom portion 28, is generally aligned over the axis of implant 60.

In certain embodiments, wherein the implant excision aid has a window located on the bottom portion of the first plate. The window reduces the chance for direct contact between the excision aid and an excision wound. The existence of a window contributes to a more sterile procedure wherein no portion of the excision aid touches the incision site. Reducing the risk of contact between the excision aid (or any device or tool) can reduce the risk of infection. Also, the window can further improve the function of the excision aid. The window can allow for the proximal end of the implant to lift without any restriction or barrier, thus better enabling the popping action of the proximal end of the implant.

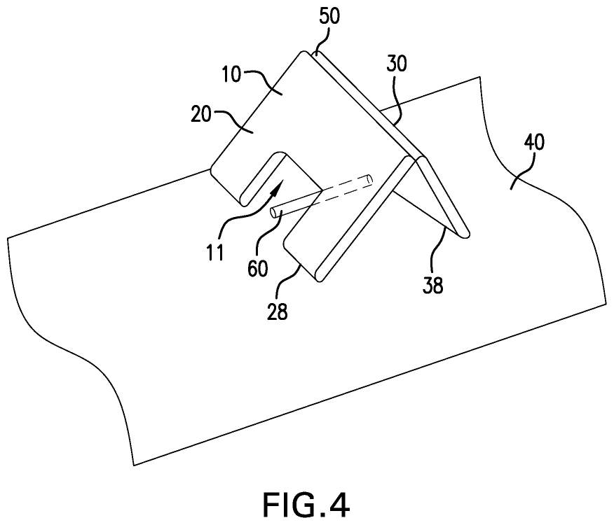

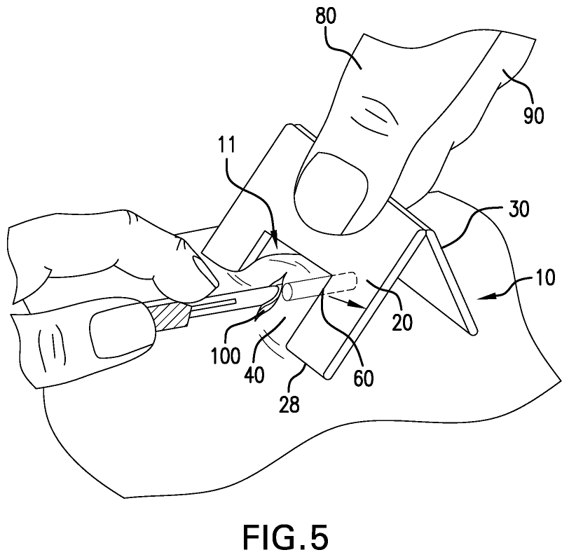

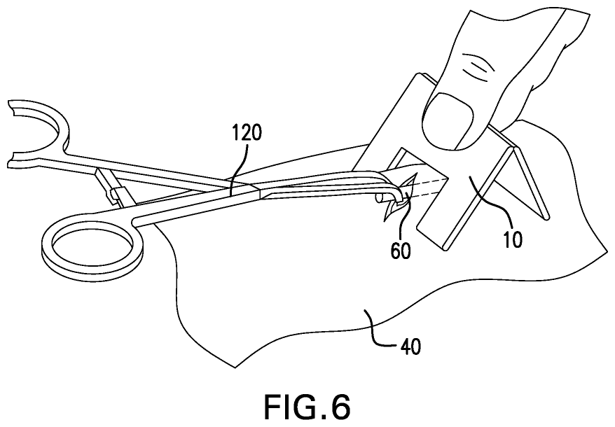

When the implant excision aid is positioned in a way that is shown in FIG. 4, the remover then can force the plates into closer proximity to one another using their thumb and forefinger. This combination of motion and force causes popping of the implant and tenting of skin between plates. This process is depicted in FIG. 5. As shown in FIG. 5, the remover uses their thumb 80 and forefinger 90 to force the first plate 20 and the second plate 30 into closer proximity to one another. Ideally, such motion causes the tenting of the skin 40 and the window 11, located at the bottom portion 28 of the first plate 20, allows the tented portion of skin 40 to be exposed. As shown in FIG. 5, an incision 100 is made in the tented region of the skin 40, whereby the incision exposes the end of implant 60. As shown in FIG. 6, while force is still being applied to the implant excision aid 10, implant 60 is then removed using an appropriate instrument, such as forceps 120. After the removal of implant, the implant excision aid is removed from skin 40 and the incision is closed.

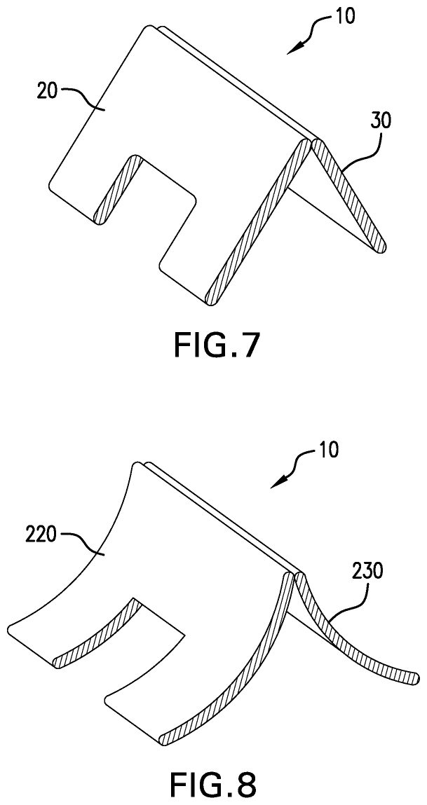

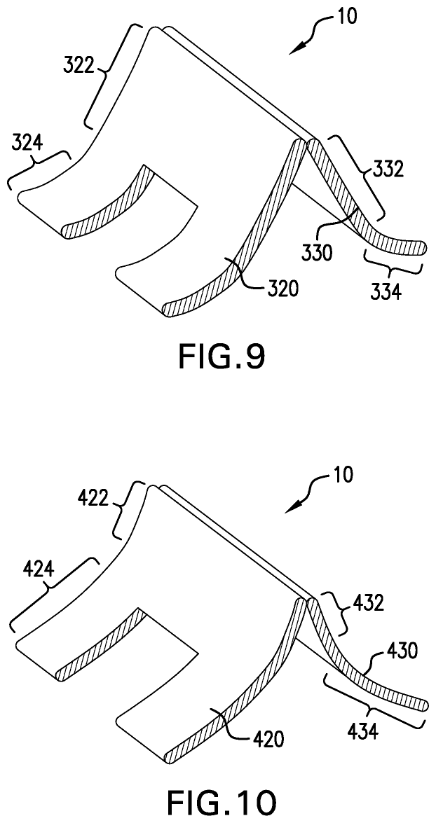

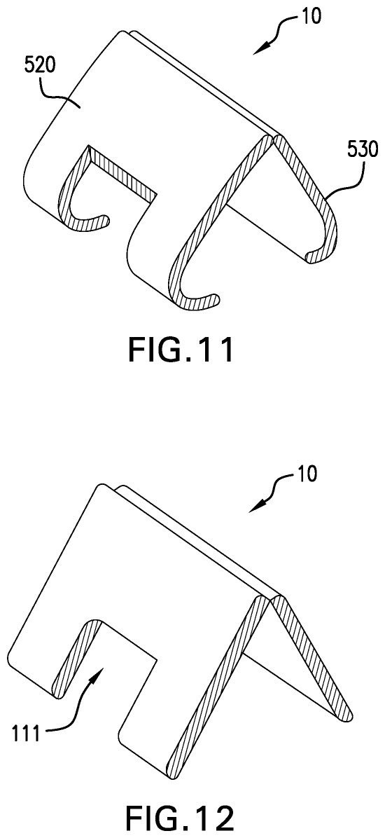

As described herein, the excision aids have a first and second plate. In certain embodiments of the excision aids described herein and shown in FIG. 7, the first plate 20 and the second plate 30 comprise generally planer shapes. FIG. 8 shows an alternate embodiment, wherein the first plate 220 and the second plate 230 comprise curved surfaces. FIG. 9 shows yet another embodiment, wherein the first plate and the second plate comprise a combination of planar surfaces 322 and 332 and curved surfaces 324 and 334. FIG. 10 shows yet another embodiment, wherein the first plate and the second plate comprise a combination of planar surfaces 422 and 432 and curved surfaces 424 and 434. In certain embodiments, the direction of curvature of the first plate 420 and second plate 430 is such that the curved surfaces of the plates that face each other are convex. However, it may be desirable in specific applications that the first plate 520 and second plate 530 are concave, as shown in FIG. 11.

Depending on the nature of the specific application for excision, including size of the patient and the location of implant, the implant excision aid may comprise a range of sizes, ranging from 15 mm to 100 mm in height and 15 mm to 75 mm in width. In certain embodiments, the implant excision aid may be about 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm, 75 mm, 76 mm, 77 mm, 78 mm, 79 mm, 80 mm, 81 mm, 82 mm, 83 mm, 84 mm, 85 mm, 86 mm, 87 mm, 88 mm, 89 mm, 90 mm, 91 mm, 92 mm, 93 mm, 94 mm, 95 mm, 96 mm, 97 mm, 98 mm, 99 mm or 100 mm in height, measuring from the top of the first or second plate to the bottom of the first or second plate.

In certain embodiments, the implant excision aid may be about 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, 50 mm, 51 mm, 52 mm, 53 mm, 54 mm, 55 mm, 56 mm, 57 mm, 58 mm, 59 mm, 60 mm, 61 mm, 62 mm, 63 mm, 64 mm, 65 mm, 66 mm, 67 mm, 68 mm, 69 mm, 70 mm, 71 mm, 72 mm, 73 mm, 74 mm or 75 mm in width, measuring from the right side of the first or second plate to the left side of the first or second plate.

In certain embodiments, wherein the excision aid contains a window located on the first or proximal plate, the width of window may comprise a range extending from 10% of the width of the first or proximal plate to 75% of the width of the first or proximal plate. In certain embodiments, the width of the window may comprise 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75% of the width of the first or proximal plate.

Similarly, the height of the window may comprise a range extending from 10% of the height of the first or proximal plate to 75% of the height of the first or proximal plate. In certain embodiments, the height of the window may comprise 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74% or 75% of the height of the first or proximal plate.

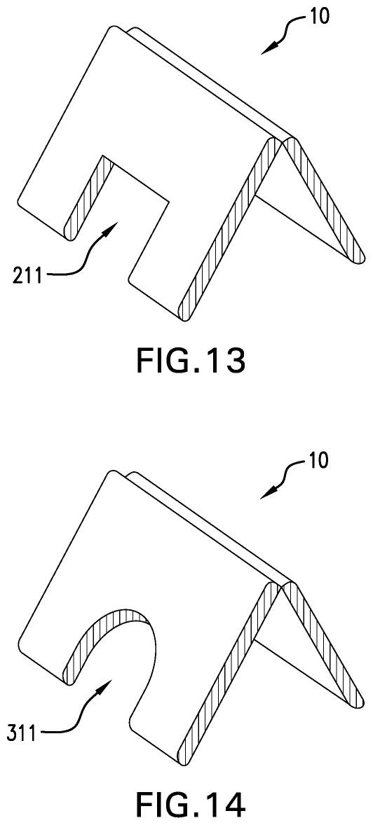

In certain embodiments of the excision aids described herein, wherein the excision aid has a window, the periphery of the window in the implant excision aid can take on any shape including, but not limited to, a quadrilateral periphery such as rectangular, square or rhombus shape or a circular periphery. In certain embodiments, the window in the implant excision aid may comprise a combination of linear and circular elements. As shown in FIG. 12, the window 111 may comprise a combination of linear and circular elements. As shown in FIG. 13, the window 211 may comprise a rectangular periphery. As shown in FIG. 14, the window 311 may comprise a circular periphery.

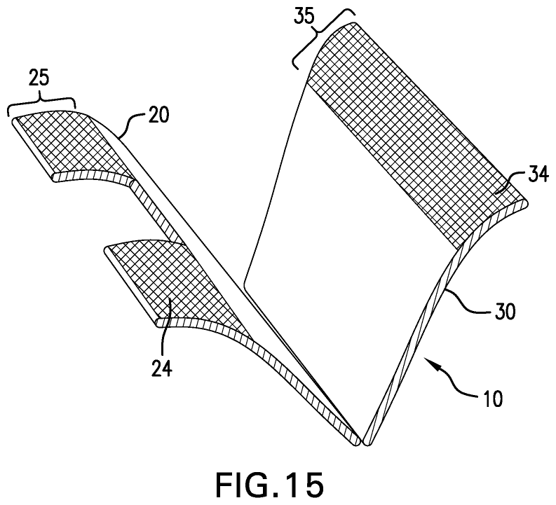

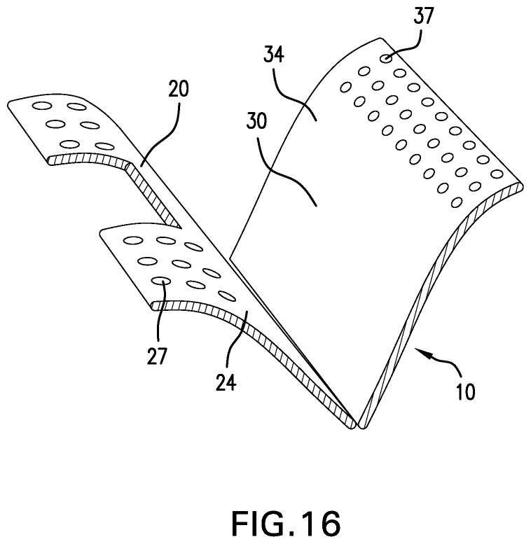

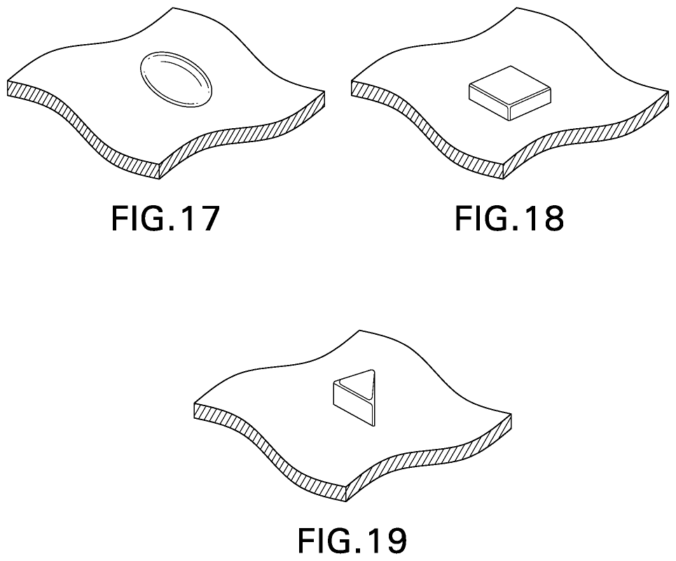

In certain embodiments of the excision aids described herein, the bottom portion of the first plate or the second plate may comprise features for aiding in gripping and moving the skin when the first plate and second plate are forced closer together. In certain embodiments of the excision aids described herein, the internal surface or the edge of either the first plate or the second plate may comprise features for aiding in gripping and moving the skin when the first plate and second plate are forced closer together. In certain embodiments of the excision aids described herein, the internal surface of the bottom portion of the first plate or the second plate may comprise features for aiding in gripping and moving the skin when the first plate and second plate are forced closer together. In the embodiment shown in FIG. 15, the internal surface 24 of the first plate 20 and the internal surface 34 of the second plate 30 of the implant excision aid 10 have a texture 25 and 35. In the embodiment shown in FIG. 16, the internal surface 24 of the first plate 20 and the internal surface 34 of the second plate 30 of the implant excision aid 10 have a pattern of protrusions 27 and 37. Viewed via a cross-section or in plain view, protrusions may have any shape that will increase the friction between the implant excision aid and the patient's skin. In certain embodiments, protrusions may be semi-circular in shape as shown in FIG. 17, rectangular in shape as shown in FIG. 18, or triangular in shape, as shown in FIG. 19. It should be obvious to anyone skilled in the art that combinations of said shapes could also be used.

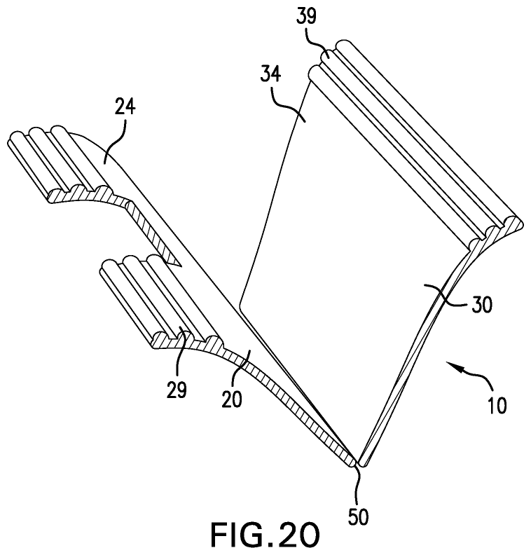

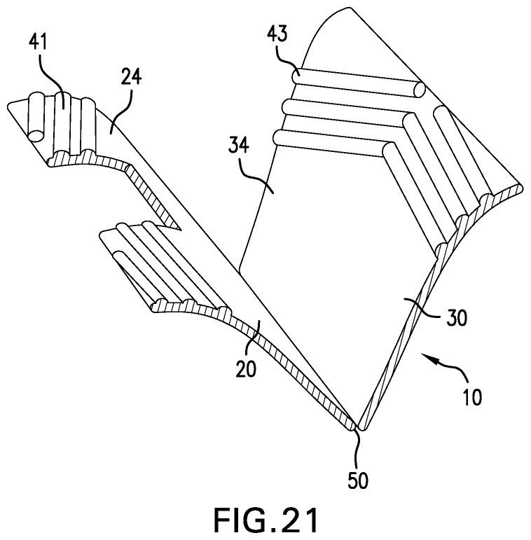

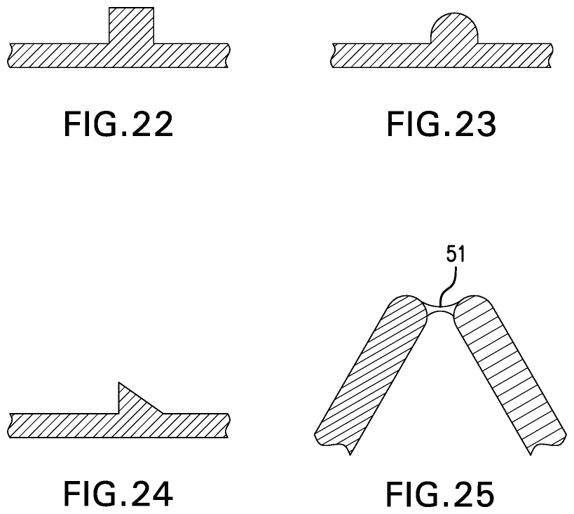

In certain embodiments of the excision aids described herein, the bottom portion of the first or second plate may include one or more ribs in various configurations. In certain embodiments of the excision aids described herein, the internal surface of the first or second plate may include one or more ribs in various configurations. In certain embodiments of the excision aids described herein, the internal surface of bottom portion of the first or second plate may include one or more ribs in various configurations. In the embodiment shown in FIG. 20, the internal surface 24 of the first plate 20 and the internal surface 34 of the second plate 30 of the implant excision aid 10 comprise one or more ribs 29 and 39 oriented parallel to the axis of the hinge 50. In the embodiment shown in FIG. 21, the internal surface 24 of the first plate 20 and the internal surface 34 of the second plate 30 of the implant excision aid 10 comprise a combination of one or more ribs 41 and 43 that are positioned parallel to adjacent ribs and oblique to the axis of the hinge 50. In other embodiments of the implant excision aids described herein, wherein the internal surface of the first plate and the second plate contain ribs, the ribs may comprise a cross-section shape that is rectangular, as shown in FIG. 22, circular, as shown in FIG. 23, triangular, as shown in FIG. 24, or any combination of these. The projection of ribs can range from 0.1 mm to 6 mm from the surface of plates.

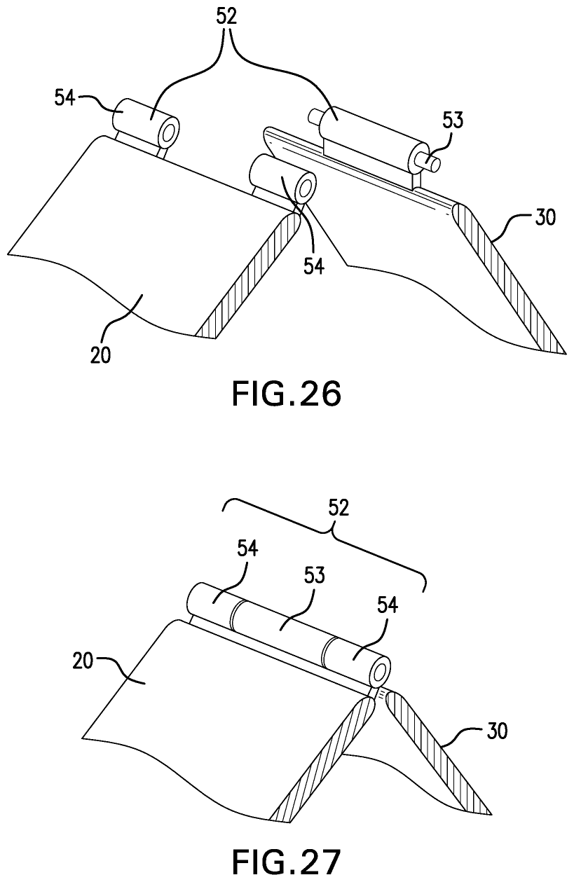

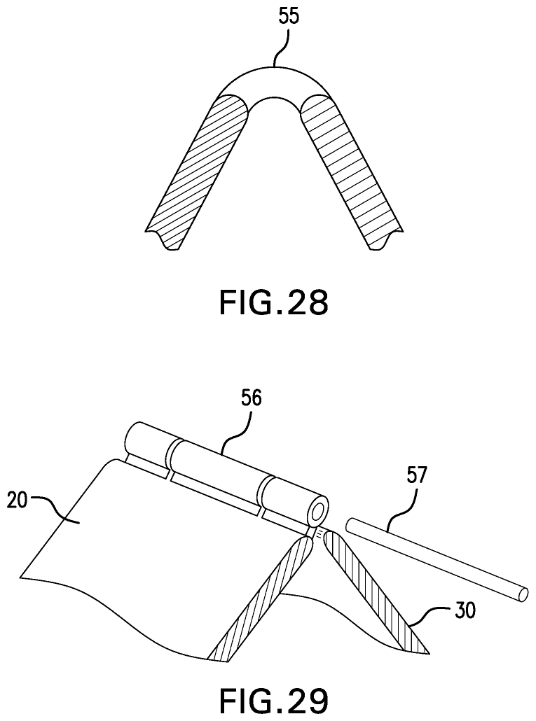

The implant excision aids described herein include a hinge connecting the top portion of the first and second plate. In certain embodiments, as shown in FIG. 25 the hinge 51 is an integrally-molded living hinge. In an alternate embodiment, FIG. 26 and FIG. 27 show a hinge 52 comprising pins 53 and receptacles 54 to form a rotating hinge between the first plate 20 and the second plate 30, which can be assembled as two separate components. Alternatively, the hinge shown in FIG. 27 may be produced by molding the first plate and the second plate in incompatible resins in a two-shot molding process, such that the two components may rotate freely relative to each other. In an alternate embodiment, as shown in FIG. 28, hinge 55 is integrally molded as a flexible component. The integrally molded hinge can be molded in a two-shot molding process, wherein the hinge comprises a third component with flexural properties, including, for example, an elastomeric element serving as a hinge. In yet another embodiment, as shown in FIG. 29, hinge 56 is integrated with the first plate 20 and second plate 30 and connected with a hinge pin 57.

In many instances, it may be difficult for the remover to consistently maintain the appropriate amount of closure or force on the implant excision aid during the excision procedure. In certain embodiments, the implant excision aid can comprise a feature that prevents it from re-opening once it has been applied to the extraction site.

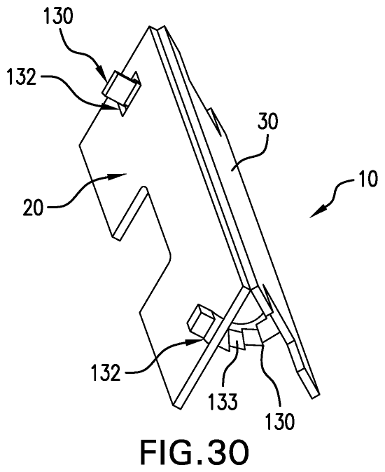

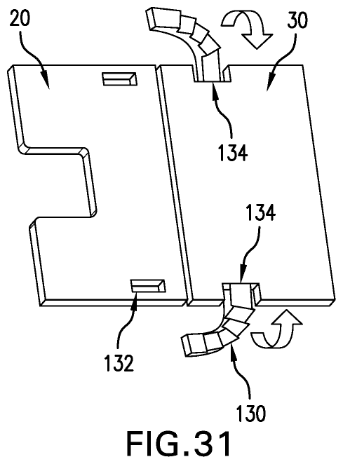

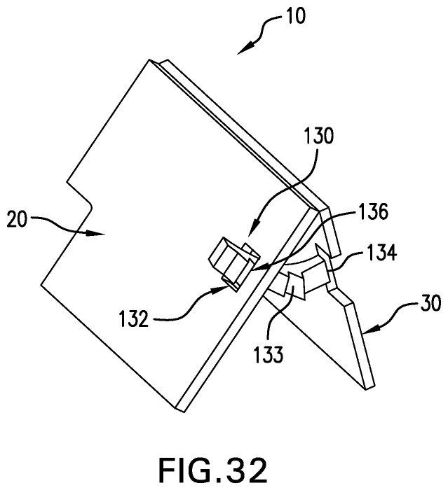

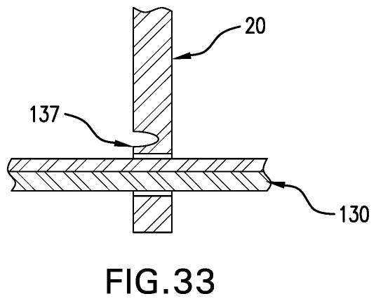

In certain embodiments of the implant excision aids described herein, the excision aids comprise a feature that maintains the correct distance between the first plate and second plate. In certain embodiments, such a feature could be a non-return feature. In certain embodiments the excision aid described herein can include a ratchet system that includes at least one ratchet arm and at least one complementary opening to receive the ratchet arm. FIG. 30 shows an embodiment of a ratchet system comprising a pair of ratchet arms 130 located on the top portion of the second plate 30 which can be directed through openings 132 in the first plate 20. The ratchet arms 130 comprise a series of ramps, or teeth, which interact with the edge of openings 132 to allow movement of the first plate 20 and the second plate 30 towards each other, but not away from each other. In certain embodiments, as shown in FIG. 31, the ratchet arms 130 may comprise living hinge features 134 for attachment to the second plate 30. A living hinge attachment allows creation of teeth by means of injection molding, and rotation of ratchet 130 into the correct orientation for insertion into the openings 132 on the first plate after molding. In certain embodiments, a living hinge is a thin flexible feature between the first and second plates, wherein the hinge feature is substantially thinner than the first plate or the second plate, wherein the hinge feature is between 0.1 mm and 0.75 mm in thickness, wherein the plates rotate relative to one another along the line of the hinge. A small residual force generated by living hinge 134 directs ratchet arms 130 outward after insertion into openings 132, causing engagement of teeth with the mating edge 136 of openings 132, as shown in the embodiment of FIG. 32. As the remover brings the first plate and the second plate into contact with skin and closes the distance between the first plate and the second plate, the ratchet arms prevent re-opening of the distance between the first plate and the second plate. Ratchet arms also enable the remover to consistently maintain the desired distance between the first plate and the second plate without having to continually hold pressure between them. The number of teeth or ramps can vary depending on requirements specific to the application. The number of teeth or ramps on a ratchet arm may range between one and 15. In certain embodiments, the number of teeth or ramps on a ratchet arm can be 1, 2, 3, 4, 5, 6, 7, 8, 9, 10, 11, 12, 13, 14 or 15. Alternatively, ratchet arms may be located on the first plate and the openings may be located on the second. In an alternate embodiment, teeth may be replaced by a resilient planar surface on ratchet 136, which interacts with an inclined blade 137 to allow relative motion in only one direction, as shown in FIG. 33.

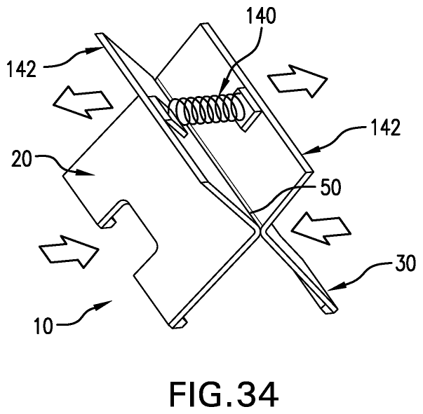

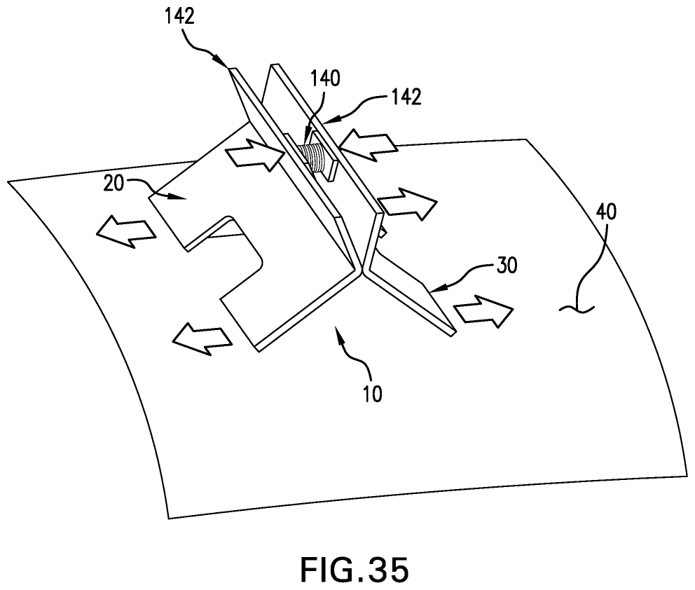

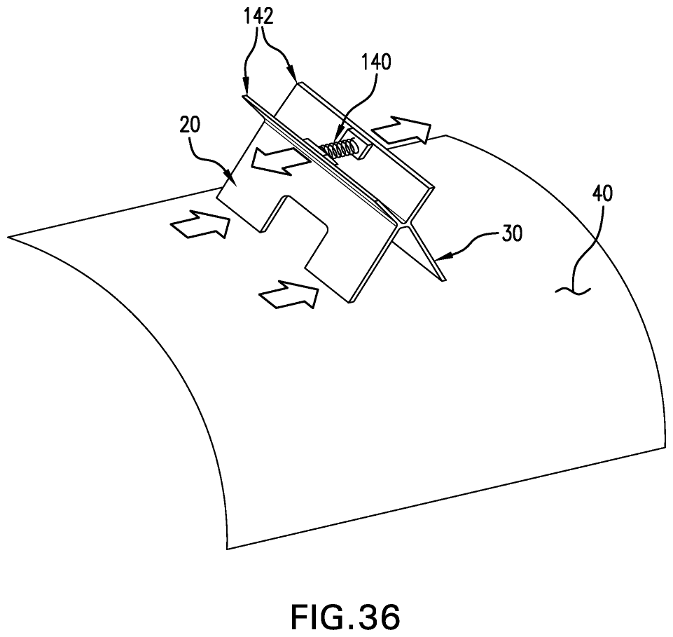

FIG. 34 shows an alternate embodiment, the implant excision aid 10 comprises a coil-type compression spring 140 that forces the first plate 20 and the second plate 30 together. In FIG. 34, the implant excision aid further comprises handles 142 located on the opposite sides of hinge 50 from the first plate 20 and second plate 30. To operate, as shown in FIG. 35, handles 142 are squeezed together to open the first plate 20 and the second plate 30 for application to skin 40 in the excision area. Once the implant excision aid is located properly on skin 40, the remover releases handles 142 so that spring 140 can exert the appropriate pinching force in the excision area, as shown in FIG. 36. It is expected that some force may still need to be applied to hold the implant excision aid in contact with skin, but the spring would minimize the force required to hold the first plate and the second plate in the “pinch” position.

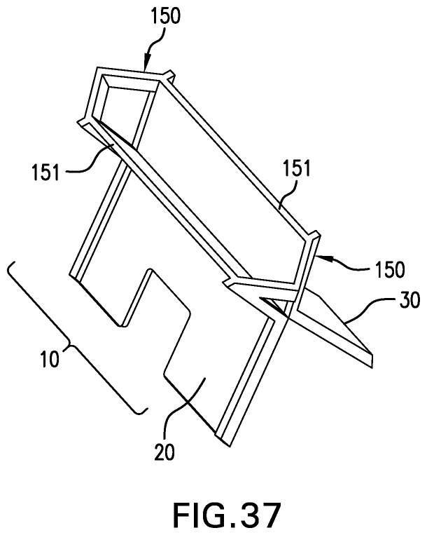

FIG. 37 shows an alternate embodiment. In FIG. 37 springs 150 can be integrally formed in the implant excision aid 10. Integral springs 150 generally function as leaf springs that deform as handles 151 are squeezed together. Energy stored in springs 150 is released to create a return force that forces the first plate 20 and the second plate 30 closer together when handles 151 are released.

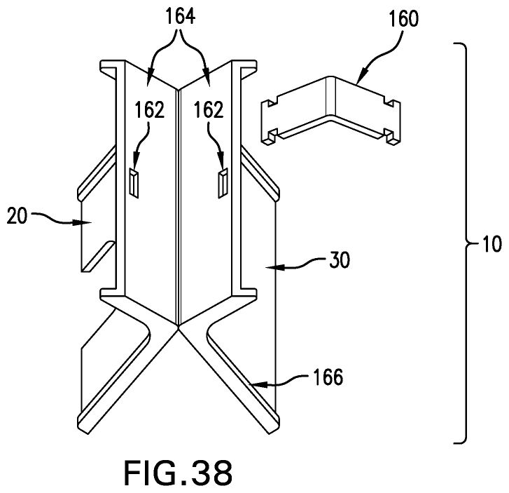

FIG. 38 shows an alternate embodiment, in FIG. 38 the implant excision aid 10 comprises a separate spring 160 that is snapped into place in receptacles 162 in handles 164 of the implant excision aid 10. The implant excision aid 10 may comprise stiffening ribs 166 at various places on the first plate 20 and the second plate 30 to enable transfer of closure force generated by spring 160 to the edges of the first plate and the second plate without undesirable bending of the first plate and second plate. Separate spring 160 functions to force closure between the first plate 20 and the second plate 30. Separate spring 160 may be configured to create a return force appropriate for the application by altering its length, cross-section, and material.

Also disclosed herein are methods for aiding in the removal of a subdermal or subcutaneous implant using the implant excision aid described herein. In certain embodiments, the method for aiding in the removal of a subdermal or subcutaneous implant using the implant excision aid described herein includes the steps of contacting the bottom portion of the first and second plate with the patient's skin, wherein the majority of the implant is located between the internal surfaces of the first and second plate of the excision aid; exerting a force on the excision aid to move the skin in the distal direction and/or to locally displace the proximal end of the implant toward the skin surface resulting in the tenting of skin external to the implant to facilitate creating an incision in the skin and extracting the implant.

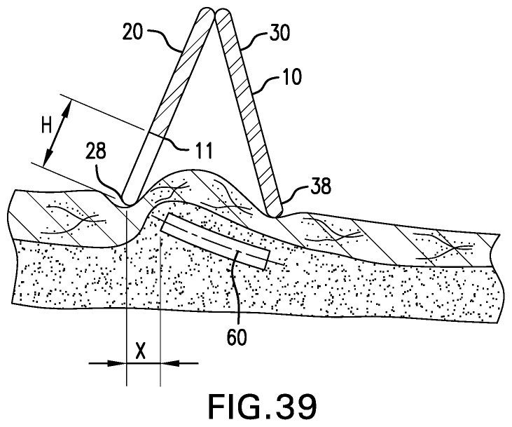

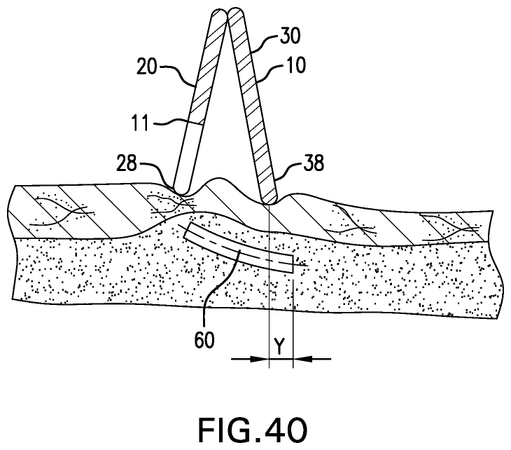

In certain embodiments, wherein the implant excision aid has a window, the bottom portion of the first plate is placed adjacent to the proximal end of the implant or, alternatively; the bottom portion of the first plate is placed in front of the proximal end of the implant. In such an embodiment, the bottom portion of the second plate is placed at the distal end of the implant to provide a backstop for the implant or, alternatively; the second plate is placed on top of the distal end of the implant so that a vertical force can be applied to the distal end of the implant to further facilitate the popping of the proximal of end of the implant. FIG. 39 shows a method of using an excision aid 10 described herein, wherein the implant excision aid has a window 11 and the bottom portion 28 of the first plate 20 is placed in front of the proximal end of the implant 60 by a distance of “x” and the bottom portion 38 of the second plate 30 is placed on top of the distal end of the implant 60 so that a vertical pressure can be applied to the distal end of the implant to further facilitate the popping of the proximal of end of the implant. FIG. 40 shows a method of using an excision aid 10 described herein, wherein the implant excision aid has a window 11 and the bottom portion 28 of the first plate 20 is placed adjacent to the proximal end of the implant 60 and the bottom portion 38 of the second plate 30 is placed on top of the of the implant 60 a distance of “y” from the distal end so that a vertical pressure can be applied to the distal end of the implant to further facilitate the popping of the proximal of end of the implant.

In certain embodiments, wherein the implant excision aid does not include a window, the bottom of the first plate is placed adjacent to proximal end of the implant or, alternatively; the bottom of the first plate is placed just behind the proximal end of the implant. In such an embodiment, the bottom of the second plate is placed at the distal end of the implant to provide a backstop for the implant or, alternatively; the second plate is placed on top of the distal end so that a vertical pressure can be applied to the distal end of the implant to further facilitate the popping of the proximal of end of the implant.

Also disclosed herein are processes of making and manufacturing the implant excision aids described herein. Described herein are processes of manufacturing the implant excision aid described herein wherein the implant excision aid made by injection molding and the implant excision aid is an injection molded part. The implant excision aid described herein can also be molded in structural foam, machined from plastic or fabricated from metal. Suitable plastics may include, but are not limited to, polypropylene, polyethylene, ABS, nylon, polyester, and POM. Suitable metals include, but are not limited to, stainless steel and aluminum. In an alternate embodiment, the implant excision aid can be 3D printed using a 3D printer.

What is claimed is:

1. An implant excision aid for removing a subdermal or subcutaneous implant from a patient comprising:
a first plate comprising an external surface, an internal surface, a top portion, a bottom portion, a right side and a left side, wherein the bottom portion of the first plate is in communication with the patient's skin during use;
a second plate comprising an external surface and an internal surface, a top portion, a bottom portion, a right side and a left side, wherein the bottom portion of the second plate is in communication with the patient's skin during use, wherein at least a portion of the bottom portion of the first or second plate or the internal surface of the first plate or second plate comprises a polymer or a metal, wherein the polymer or the metal generates enough friction so that the portion of the bottom portion of the first or second plate or the internal surface of the first plate or second plate is capable of moving the patient's skin during use; and
a hinge wherein the hinge connects the top of the first plate to the top of the second plate.

2. The implant excision aid of claim 1, wherein the first plate further comprises a window located at the bottom of the plate.

3. The implant excision aid of claim 1, wherein at least a portion of the internal surface of the first plate or second plate further comprises a textured surface, wherein the texture on the surface increases the friction between the bottom portion of the first or second plate or the internal surface of the first plate or second plate and the patient's skin during use.

4. The implant excision aid of claim 1, wherein the first and the second plate are generally planar.

5. The implant excision aid if claim 1, wherein the first and the second plate are generally curved.

6. The implant excision aid of claim 1, wherein the first and the second plate independently comprise a combination of planar and curved elements.

7. The implant excision aid of claim 3, wherein the textured surface located on at least a portion of at least the internal surface of the first plate or second plate comprises a pattern of protrusions.

8. The implant excision aid of claim 3, wherein the textured surface located on at least a portion of the internal surface of the first plate or second plate comprises one or more ribs.

9. The implant excision aid of claim 8, wherein the ribs are in a pattern that is parallel to the hinge.

10. The implant excision aid of claim 8, wherein the ribs are in a pattern that is oblique to the hinge.

11. The implant excision aid of claim 8, wherein the ribs are in a pattern comprising a combination of oblique and parallel orientations relative to the hinge.

12. The implant excision aid of claim 8, wherein the ribs are curved.

13. The implant excision aid of claim 1, wherein the hinge is a living hinge.

14. The implant excision aid of claim 1, wherein the hinge is an integral hinge pin and a receptacle.

15. The implant excision aid of claim 1, wherein the hinge comprises an integrally molded flexible element.

16. The implant excision aid of claim 1, wherein the hinge comprises a hinge pin connecting the first and second plates.

17. The implant excision aid of claim 1, further comprising a ratcheting system, wherein the ratcheting system comprises at least one ratcheting arm located on the upper portion of the first or second plate, wherein each ratcheting arm comprises at least one tooth; and
at least one opening located on the upper portion of the opposing plate, wherein the opening is capable of receiving the ratcheting arm as the ratcheting arm moves forward and wherein, the tooth on the ratcheting arm interacts with the edge of the opening to prevent the ratcheting arm from moving backwards.

18. The implant excision aid of claim 1, wherein the implant excision aid comprises polypropylene.

19. The implant excision aid of claim 1, further comprising a coil-type compression spring connecting the first and second plate.

20. The implant excision aid of claim 1, further comprising one or more of leaf springs connecting the first and second plate.

21. A method for aiding in the removal of a subdermal or subcutaneous implant using the implant excision aid of claim 1, comprising contacting the bottom portion of the first and second plate with the patient's skin, wherein the majority of the implant is located between the internal surfaces of the first and second plate of the excision aid; exerting a force on the excision aid to cause tenting of skin external to the implant, wherein the excision aid locally displaces the proximal end of the implant toward the skin surface, while moving the skin in the distal direction to facilitate creating an incision in the skin and extracting the implant.

22. A process of making the implant excision aid of claim 1, wherein the implant excision aid is injection molded from plastics.

23. A process of making the implant excision aid of claim 1, wherein the excision aid is machined from plastics.

24. A process of making the implant excision aid of claim 1, wherein the implant excision aid is machined from metal.

25. A process of making the implant excision aid of claim 1, wherein the implant excision aid is printed using a 3D printer.
